(19) United States
(12) Patent Application Publication
(10) Pub. No.: US 2025/0212921 A1
(43) Pub. Date: Jul. 3, 2025

(54) Fermentation Of Cashew Nuts by Koji for an Animal Free Ersatz of Foie Gras

(71) Applicant: DR. FOODS CO., LTD, Tokyo (JP)

(72) Inventor: Alexis Guionet

(73) Assignee: DR. FOODS CO., LTD, Tokyo (JP)

(21) Appl. No.: 18/852,367

(22) PCT Filed: Mar. 31, 2023
(86) PCT No.: PCT/JP2023/013716

§ 371 Date: Sep. 27, 2024

(30) Foreign Application Priority Data
Apr. 1, 2022 (JP) 2022-061974

(51) Int. Cl. A23L 25/00; A23L 31/00

(52) U.S. Cl. CPC A23L 25/40; A23L 31/00

(57) ABSTRACT

A process for fermenting nuts or beans (or a mixture of nuts and beans) with fungus. The process of making an animal-free “foie gras” substitute is described, in which nuts or beans are fermented by fungi to mimic the taste of liver without the need for miso, soy sauce, yeast extract, or other separately fermented materials.

### TECHNICAL FIELD

The invention relates to the manufacture of a nut-based paste similar in taste and appearance to the French food specialty called “foie gras”. The plant-based “foie gras” produced according to this invention provides a strong umami and liver flavour without the need for miso, soy sauce or yeast extract. The undesirable taste of starch is also reduced. The sugar released by digesting the starch is partially consumed by the fungi; the remaining part replaces the sugar added when seasoning the real “foie gras” (directly as powdered sugar or by seasoning with dessert wine). The fungi are richer in protein than in starch, just like the real foie gras, which help to imitate its taste.

### BACKGROUND ART

Foie gras is a French specialty, expensive and generally considered delicious. It is produced by geese or ducks that are force-fed in such a way that their livers accumulate fat to the point of becoming adipose (hepatic steatosis). Even setting aside vegetarians, and even though this phenomenon may occur naturally in migratory birds, this process sometimes appears controversial, as more and more people are concerned about animal welfare. In addition, the process is expensive and has a relatively high carbon footprint, due to the overfeeding of the geese or ducks. Therefore, vegetable pastes that attempt to mimic the texture and taste of real foie gras have been developed.

Animal-free ersatz foie gras, also called “vegan foie gras” or “faux gras”, is usually based on a mixture of cashews nuts (for taste and texture properties); fermented materials such as miso, soy sauce or yeast extract (for umami and liver flavour); cocoa butter and coconut oil (for texture capable of melting at 40° C.); and alcohol, salt, pepper and spices for seasoning as in real foie gras. These types of “vegan foie gras” contain too much starch (from cashews nuts) and not enough protein compared to real foie gras, which makes the taste slightly different from real foie gras.

### SUMMARY OF INVENTION

### Technical Problem

This patent describes a new method of making an animal-free “foie gras” substitute, in which nuts or beans are fermented by fungi to mimic the taste of liver without the need for miso, soy sauce, yeast extract, or other separately fermented materials.

### Solution to Problem

Both the gel medium used to maintain the fungal strain on a petri dish and the liquid medium used to pre-culture the fungi are prepared from animal-free edible materials. Thus, both the preparation process and the production process are vegetarian.

### Advantageous Effects of Invention

The liquid pre-culture is helpful to allow uniform and vigorous development of the fungi in the nuts or beans. But commercially available spore can also be used, by spreading them evenly on the cashew nuts. The fungi are grown on the sterilized (autoclaved) nuts or beans as a substrate, with controlled humidity and temperature.

It generates various transformations of the raw material: 1—Fungi digest part of the starch of nuts or beans by secreting amylase. The glucose released by the enzymatic digestion is absorbed by the fungi. Thus, the starch concentration is reduced but the glucose concentration remains low. 2—Fungi secrete glutaminase (especially Aspergillus, but Penicillium as well), an enzyme also presents in the liver of vertebrates, which is responsible for the conversion of glutamine into glutamate. By secreting glutaminases and proteases, Aspergillus and Penicillium release glutamate, which helps mimic the flavours of the liver. Glutamate is also responsible for umami, which is why our fermented nuts have more umami than unfermented nuts. 3—As fungi, Aspergillus and Penicillium synthesizes a large amount and wide variation of protein during its growth, but synthesizes very small amount of carbohydrates and lipids. Among eukaryotes, Aspergillus oryzae is even known to have the highest capacity for protein secretion. As a result, the protein content and meatiness of the product increases. 4—Since koji is a fermented food that provides prebiotics (e.g. glycosylceramides) and other good nutrients, the health food image of the final product is enhanced. 5—Other enzymes and secondary metabolites are produced. The proportion of each enzyme and metabolite is determined by the incubation temperature. By default, a fairly high temperature (>35° C.) is used, as it favours the secretion of amylase, so starch digestion is optimized. However, if more umami is desired, protease can be promoted by reducing the temperature up to 25° C.

After a certain period of incubation, sterilization is carried out by autoclaving or any other thermal process suitable for industrial production. During the autoclave/thermal process, the temperature is momentarily between 50° C. and 60° C., which overactivates the enzymes produced by the fungi (amylases, glutaminase, etc.), digesting more starch and releasing more glutamate. The whole process then ensures inactivation of the enzymes, as well as a complete inactivation (death) of the fungi and any other microorganism potentially present. This ensures a longer shelf life of the final product. Heating also accelerates the Maillard reaction (glycation) which binds the simple sugars released by the amylases to peptides and amino acids released by proteases. A sa result, the sweetness is reduced and the taste of the meat is increased.

After cooling, the material is then mixed and mashed with various vegetable oils and butters (for texture), spices, salt, pepper and alcohols such as cognac, fine, marc, armagnac, etc (for flavour), and beet root, tomato concentrate or other natural pigments (for colour). The umami taste can be adjusted by adding other fermented foods such as soy sauce, miso or yeast extract (if the taste is too weak) or by adding unfermented nuts (if the taste is too strong). The word “fermentation” should be taken in its vernacular acceptation: it refers to the process using micro-organisms to transform an edible material. It actually includes all metabolisms such as those scientifically known as “aerobic respiration”, “anaerobic respiration”, “aerobic fermentation” or “anaerobic fermentation”.

### BRIEF DESCRIPTION OF DRAWINGS

FIG. 1—Appearance of cashews incubated without and with koji.

FIG. 2—Schematic of the manufacturing principle.

### DESCRIPTION OF EMBODIMENTS

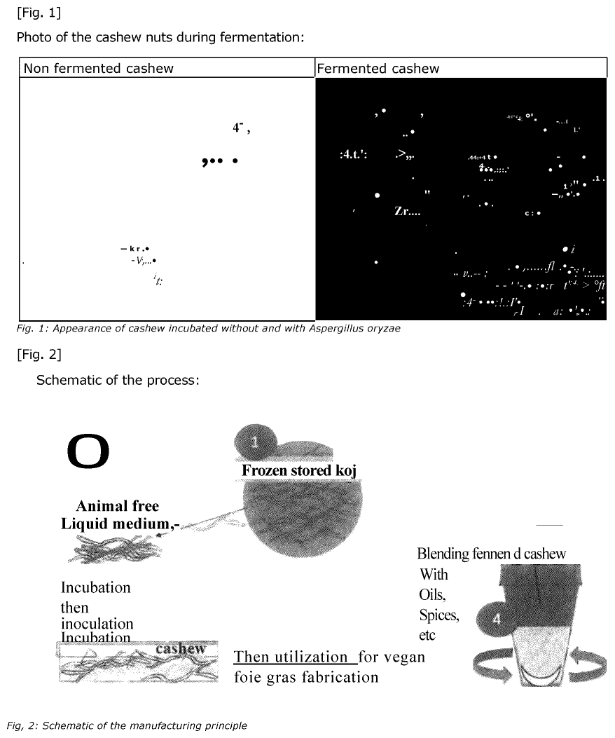

FIG. 1: Appearance of cashews incubated without and with koji. Photos are taken 1 day after the liquid culture was added to the cashews. The photo on the left shows the culture medium-cashew nut mixture in the control sample (the koji was not inoculated into the liquid culture medium). The right photograph shows the culture medium-cashew mixture in the inoculated sample (the koji was inseminated into the liquid culture medium). The hyphal structure is only visible in the right photograph, confirming a good development of the koji in the culture medium cashew mixture. This figure proves the effectiveness of the koji culture protocol on cashew nuts.

FIG. 2: Schematic of the manufacturing principle. The diagram represents the steps of the manufacturing process. 1—The koji strain is kept frozen on a solid plant medium, with periodic transplantation to a new solid plant medium. 2—This koji is used to inseminate the liquid plant medium for a determined time at a determined temperature. 3—The koji grown in the liquid medium is incubated with the sterilized cashew nuts for a specified time at a specified temperature. All the material is then autoclaved or heat sterilized. 4—After cooling, it is blended, mixed and textured with vegetable fat, seasoned and coloured. 5—Finally, it can be canned for sale.

Example: Step 1: preparation of liquid koji culture: 100 mL of soy milk, 100 ml of water and 4 g of corn starch are mixed together. After autoclaving and cooling down, few spore or hyphae of Aspergillus oryzae are put in the mixture. It is kept at 37° C. under agitation for 2 days in order to get a con-centrated fungal culture (estimated around 10 000 CFU/mL). Step 2: preparation of the fermented cashew nuts with koji: 400 g of cashew nuts are autoclaved (121° C., 20 minutes) in a glass Petri dish. After cooling down, all the fungal liquid mixture prepared 2 days before are fill in the dish in such a way to cover the cashew nuts. After few days of incubation at 37° C., the fermented cashew is autoclaved (121° C., 20 minutes). Step 3: cooking the cashew-koji: 350 g of fermented cashew (as explained in step 1 and 2) is mixed with 50 g of melted deodorized cocoa butter, 50 g of deodorized coconuts oil, 30 mL of water, 30 mL of strong aged alcohol, salt, pepper and spices. The mixture is put in jar and reserved in 4° C.

### COMPARATIVE EXAMPLE

The procedure described as above was also done without the Koji insemination step in the “soy milk-water-corn starch” broth, to get a plant based non-fermented foie gras. A panel of 5 people ate both the plant-based foie gras made with fermented cashew nuts (as explained in the paragraph “example”) and the plant-based foie gras made with non-fermented cashew nuts. Meanwhile, a real foie gras made from goose liver in Strasbourg by a traditional method was been ate for comparison. Everyone in the panel found the plant based fermented foie gras more similar to the real foie gras than the plant based non-fermented foie gras, in terms of taste and colour.

What is claimed is:

1-14. (canceled)

15. A method if incubating cashew nuts with fungi comprising
using a pre-culture of fungi grown in plant-based broth or;
using of commercially available spores that can be spread directly on the cashew nuts.
